(19) United States
(12) Patent Application Publication
(10) Pub. No.: US 2026/0366047 A1
(43) Pub. Date: Nov. 19, 2026

(54) METHODS AND COMPOSITIONS FOR THE PRODUCTION OF GLYOXAL

(71) Applicant: Solugen, Inc., Houston, TX (US)

(72) Inventors: Zhizeng Sun, Houston, TX (US); Brian F. Fisher, Houston, TX (US); Shuai Qian, Houston, TX (US); Lynn H. Su, Houston, TX (US); Toni M. Lee, Missouri City, TX (US); Gaurab Chakrabarti, Houston, TX (US); Sean Hunt, Houston, TX (US)

(73) Assignee: Solugen, Inc., Houston, TX (US)

(21) Appl. No.: 19/487,312

(22) PCT Filed: May 31, 2024
(86) PCT No.: PCT/US2024/032051

§ 371 Date: Nov. 24, 2025

Related U.S. Application Data
(60) Provisional application No. 63/505,511, filed on Jun. 1, 2023.

(51) Int. Cl. C12P 7/24

(52) U.S. Cl. CPC C12P 7/24

(57) ABSTRACT

A chemoenzymatic process comprising contacting ethylene glycol with a biocatalyst in the presence of oxygen under conditions suitable for the formation of glycolaldehyde, glyoxal or combinations thereof. A method of producing glyoxal comprising contacting ethylene glycol with an oxidase selected from the group consisting of a copper radical oxidase, an alcohol oxidase, a glycerol oxidase, and combinations thereof in the presence of oxygen and a catalase under conditions suitable for the formation of a product mixture comprising glycolaldehyde, glyoxal or combinations thereof.

### CROSS-REFERENCE TO RELATED APPLICATIONS

This application is a 35 U.S.C. § 371 national stage application of PCT/US2024/032051 filed May 31, 2024 and entitled “Methods and Compositions for the Production of Glyoxal,” which claims benefit of U.S. Provisional Application No. 63/505,511 filed Jun. 1, 2023 and entitled “Methods and Compositions for Production of Glyoxal,” each of which is hereby incorporated herein by reference in its entirety for all purposes.

### REFERENCE TO SEQUENCE LISTING

The instant application contains a Sequence Listing which has been submitted electronically in XML file format and is hereby incorporated by reference in its entirety. Said XML file, created on May 24, 2024, is named “23FRM002-PCT_3416-15102.xml” and is 4.096 kilobytes in size.

### TECHNICAL FIELD

The present disclosure relates generally to compositions and methods for the production of value-added chemicals. More particularly, the present disclosure relates to chemoenzymatic methods for the conversion of ethylene glycol to glyoxal.

### BACKGROUND

Glyoxal, also referred to as ethanedial or diformyl is the simplest dialdehyde. Glyoxal is used only as an aqueous solution at concentrations between about 30% and about 50%. Evaporation of this solution produces polyglyoxal, a white, infusible solid which can be depolymerized by heating. In 40% aqueous solution, glyoxal exists mainly as a hydrated monomer, dioxolate dimer, and cis and trans bis(dioxolane) trimers. These components represent 80% of the total composition with the hydrated trimer as the most abundant. The trimer is responsible for the cloudiness of the solution and crystalline precipitates in aqueous solutions. More highly condensed oligomers are also present and glycolates may also form under acidic conditions.

Glyoxal can be used to crosslink materials in a variety of ways. In the presence of alcohols and acidic conditions, a mixture of oligomeric acetals forms which can function as crosslinkers. When contacted with primary amines, glyoxal forms diimines which can be reduced to ethylene diamines for crosslinking isocyanates in coatings. When contacted with secondary amines, amino acetamides can be formed. When contacted with amides and urethanes, diamides and tetramides are obtained depending on the pH. These chemistries have been used to crosslink functionalized molecules such as cellulose, polyacrylamides, polyvinyl alcohol, keratin, etc. Specific examples include the use of glyoxal to produce 4,5-dihydroxy-2-imidazolidinone (DHEU) and its methylated derivative dimethylol dihydroxy ethylene urea (DMDHEU) for production of crease-resistant cotton fabrics. Glyoxal is also used for imparting wet-resistance to cardboard and to produce crosslinking agents such as glyoxal bisacrylamide and glyoxal bis(diallyl acetal).

In some conventional routes for the industrial production of glyoxal, acetaldehyde can be oxidized with nitric acid at 40 °C with a maximum glyoxal yield of 70%, which yields a product that is contaminated with acetic, formic, and glyoxylic acids and must be purified through an expensive ion-exchange resin.

In some other conventional routes, ethylene glycol is oxidized with air in the presence of dehydrogenation catalysts at temperatures ranging from 400 °C-600 °C, which yields a main impurity in the product mixture of formaldehyde, which is difficult to separate from the glyoxal.

Consequently, conventional technology requires intensive downstream processing to generate sufficiently pure glyoxal. An ongoing need exists for economically improved, lower carbon footprint methods for producing high purity glyoxal.

### SUMMARY

Disclosed herein is a chemoenzymatic process comprising contacting ethylene glycol with a biocatalyst in the presence of oxygen under conditions suitable for the formation of glycolaldehyde, glyoxal, or combinations thereof.

Also disclosed herein is a method of producing glyoxal comprising contacting ethylene glycol with an oxidase selected from the group consisting of a copper radical oxidase, an alcohol oxidase, a glycerol oxidase, and combinations thereof in the presence of oxygen and a catalase under conditions suitable for the formation of a product mixture comprising glycolaldehyde, glyoxal or combinations thereof.

### BRIEF DESCRIPTION OF DRAWINGS

For a detailed description of the aspects of the disclosed processes and systems, reference will now be made to the accompanying drawings in which:

FIG. 1 is a schematic depiction of a reaction for the conversion of ethylene glycol to glyoxal in the presence of a biocatalyst.

FIG. 2 is a schematic depiction of a reaction for the conversion of ethylene glycol to glyoxal in the presence of galactose oxidase and a small molecule activator.

FIG. 3 depicts a wavelength scan analysis of purpald assay products of ethylene glycol, glyoxal, and glycolaldehyde.

FIG. 4 is time course of glyoxal and glycolaldehyde production via a galactose oxidase mutant over seven hours.

FIG. 5 is a time course of ethylene glycol conversion via a galactose oxidase mutant over seven hours.

### DETAILED DESCRIPTION

Disclosed herein are methods and compositions for the production of glyoxal from ethylene glycol. Specifically disclosed herein are chemoenzymatic methods of generating glyoxal from ethylene glycol using an enzymatic catalyst capable of oxidizing ethylene glycol such as an ethylene glycol oxidase, a galactose oxidase, an alcohol oxidase, a glycerol oxidase, fragments thereof, variants thereof, or combinations thereof. In one or more aspects, the catalyst may take the form of a copper radical oxidase, an alcohol oxidase from a methylotrophic yeast such as Pichia pastoris, a glycerol oxidase from a filamentous fungus such as Aspergillus japonicus, or combinations thereof. Herein, a biocatalyst composition for use in the production of glyoxal from ethylene glycol are collectively termed a GLYCAT. In one or more aspects, the GLYCAT comprises one or more biocatalysts of the type disclosed herein. In one or more other aspects, the GLYCAT consists essentially of one or more biocatalysts of the type disclosed herein.

As will be understood by one of ordinary skill in the art with the benefit of this disclosure, the reaction conditions utilized for any particular biocatalyst composition (e.g., GLYCAT) disclosed herein will depend on a variety of factors. In one or more aspects, the reaction conditions disclosed herein contemplate the use of a GLYCAT in commercial scale reactions carried out in the presence of an oxygen source. In one or more aspects, the reactions disclosed herein are carried out in a bubble column reactor where oxygen is introduced under pressure to increase availability to the GLYCAT.

In such aspects, the GLYCAT may be present in amounts ranging from about 5 milligrams/liter (mg/L) to about 1000 mg/L, alternatively from about 10 mg/L to about 500 mg/L or alternatively from about 20 mg/L to about 200 mg/L, in a suitable buffered media providing a pH ranging from about 5 to about 10, alternatively from about 5.5 to about 9 and a substrate amount ranging from about 6.5 to about 8.5, alternatively from about 6.5 to about 8.5 or alternatively from about 7 to about 8. Oxygen may be introduced to the reaction to provide an oxygen pressure of equal to or less than about 500 psi, alternatively from about 50 to about 250 psi or alternatively from about 70 to about 150 psi.

In one or more aspects, a GLYCAT suitable for use in the present disclosure comprises an immobilized biocatalyst. The term immobilized biocatalyst herein refers to a biocatalyst (e.g., enzyme) that is physically confined or localized in a certain defined region of space with retention of their catalytic activities, and which can be used repeatedly and continuously. An immobilized enzyme may be associated with any suitable support such as a natural polymer (e.g., polysaccharide), a synthetic polymer (e.g., polystyrene), or an inorganic material (e.g., silica).

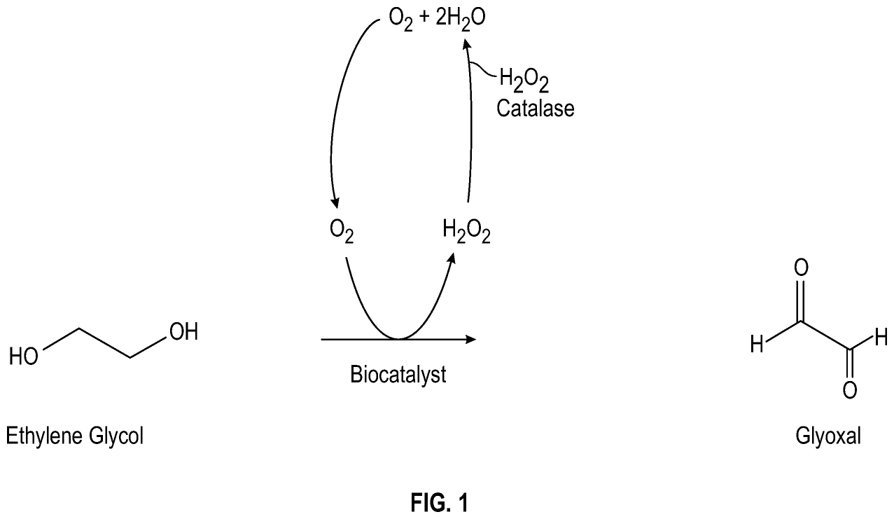

A general scheme for the production of glyoxal from ethylene glycol is depicted in FIG. 1. With reference to FIG. 1, a method of the present disclosure comprises contacting ethylene glycol with a biocatalyst composition (e.g., ethylene glycol oxidase EGOX) capable of catalyzing the oxidation of ethylene glycol to glyoxal. The reaction generates hydrogen peroxide (H₂O₂) as a by-product. Also, as depicted in FIG. 1 and as further disclosed herein, the reaction mixture may further comprise a catalase which catalyzes the disproportionation of H₂O₂.

In one or more aspects, the GLYCAT comprises a copper radical oxidase (CRO). CROs are a class of non-flavoprotein alcohol oxidoreductases that employ molecular oxygen as a terminal electron acceptor to generate hydrogen peroxide.

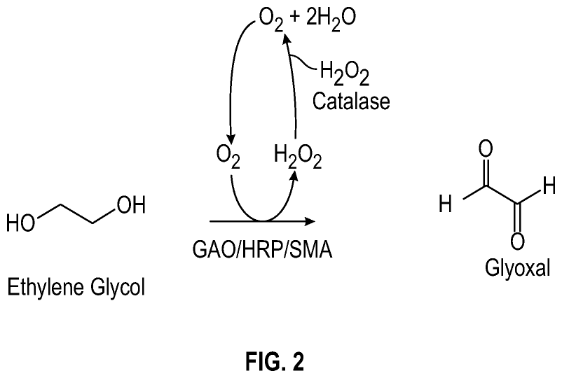

GAO is a copper enzyme secreted by some fungal species, including Fusarium graminearum (also known as Gibberella zeae), to aid in degradation of extracellular carbohydrate food sources through catalyzing the oxidation of primary alcohols to aldehydes while generating hydrogen peroxide. Not intending to be bound by theory, the native function of GAO is the oxidation of D-galactose to D-galacto-hexodialdose. A GAO suitable for use in the present disclosure has SEQ ID No: 1. In an aspect, the CRO is galactose oxidase (GAO EC 1.1.3.9) and the reaction is carried out in the presence of a small molecule activator and oxygen as depicted in FIG. 2. The small molecule activator is a cofactor that functions to rescue the activity of the GAO and is itself activated by a biocatalyst, horseradish peroxidase (HRP).

Nonlimiting examples of other small molecule activators suitable for use in the present disclosure include L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3(4H)-one, 2-oxindole, beta-lapachone, menaquinone, thiamine, 4-methyl-5-thiazoleethanol, disodium 6-hydroxy-5-((2-methoxy-5-methyl-4-sulfophenyl) azo)-2-naphthalenesulfonate (Allura Red AC), menadione, p-cresol, ethyl-[4-[[4-[ethyl-[(3-sulfophenyl)methyl]amino]phenyl]-(4-hydroxy-2-sulfophenyl)methylidene]-1-cyclohexa-2,5-dienylidene]-[(3-sulfophenyl)methyl]azanium (Fast green FCF), disodium α-(4-(N-ethyl-3-sulfonatobenzylamino)phenyl)-α-(4-N-ethyl-3-sulfonatobenzylamino)cyclohexa-2,5-dienylidene) toluene-2-sulfonate (Brilliant Blue FCF), methylisothiazolinone, caffeine, veratryl alcohol, fluorescein, and combinations thereof.

With reference to FIG. 2, the reaction of ethylene glycol results in the formation of an aldehyde intermediate, glycolaldehyde (not shown), which is further oxidized by the GLYCAT to form glyoxal.

In an aspect, the GLYCAT comprises an alcohol oxidase (AOX, EC 1.1.3.13). In another aspect, an immobilized AOX with catalase is used to oxidize ethylene glycol to the corresponding aldehyde. Nonlimiting examples of sources of an AOX suitable for use in the GLYCAT include Achatina achatina, Achatina fulica, Arion ater, Aspergillus ochraceus Aspergillus ochraceus AIU 031, Aspergillus nidulans, Aspergillus terreus Aspergillus terreus MTCC 6324, Basidiomycota Basidiomycota B191039, Byssochlamys spectabilis Byssochlamys spectabilis RI017 Paecilomyces variotii, Candida boidinii, Candida methanolica, Candida koshuensis, Candida olivarium, Candida ooitensis, Candida queretana, Candida silvicola Hanensula alcoolica, Kloeckera boidinii, Torulopsis enokii, Candida cariosilignicola, Candida guilliermondii, Pichia guilliermondii, Yamadazyma guilliermondii, Endomyces guilliermondii, Candida methanolovescense, Ogatea minuta, Candida methanosorbosa, Candida methanosorbosa M-2003, Candida sithepensis, Candida sonorensis, Torulopsis sonorensis, Candida sp. (in: Saccharomycesles), Candida sp. (in: Saccharomycesles) 25-A, Candida succiphila, Candida tropicalis, Comamonas sp., Comamonas sp. UVS, Gloeophyllum trabeum, Hansenula polymorpha, Ogataea polymorpha, Pichia angusta, Hanensula angusta, Ogataea angusta Ogataea angusta DL-1, Ogataea angusta NCYC 495, Helix aspersa, Kuraishia capsulata, Lachnellula arida, Lachnellula cervina, Lachnellula occidentalis, Lachnellula subtilissima, Lachnellula suecica, Lachnellula willkommii, Methylococcus capsulatus, Methylophilus methylotrophus, Ochrobactrum sp., Ochrobactrum sp. AIU 033, Ogataea glucozyma, Ogataea henricii, Ogataea methanolica, Pichia pinus, Ogataea minuta, Ogataea naganishii, Ogataea philodendri, Ogataea pignaliae, Ogataea pini, Ogataea siamensis, Ogataea trehalophil, Ogataea wickerhamii, Passalora fulva, Penicillium chrysogenum, Penicillium purpurascens, Penicillium purpurascens AIU 063, Phanerochaete chrysosporium, Phanerochaete chrysosporium DSMZ 1547, Phanerochaete chrysosporium K-3, Phlebiopsis gigantea, Pichia pastoris, Komagataella pastoris, Komagataella phaffil, Komagataella pseudopastoris, Endomyces pastoris, Petasospora pastoris, Zygosaccharomyces pastoris, Zygowillia pastoris, Zymopichia pastoris, Komagataella pastoris GS115, Komagataella pastoris IFP 206, Komagataella pastoris X33, Pichia putida, Polyporus obtusus, Poria contigua, Radulodon casearius, Thodotorula toruloides, Thermoascus aurantiacus, Thermoascus aurantiacus NBRC 31693, and Trametes cinnabarina. In one or more aspects, the AOX is sourced from Phanerochaete chrysosporium.

In one or more aspects, the GLYCAT comprises a glycerol oxidase (GlyOX, EC 1.1.3.9). For example, a GlyOX enzyme may have been endogenously expressed and characterized for activity on ethylene glycol. In one or more aspects, the GlyOX is an endogenous glycerol oxidase produced from Aspergillus japonicus. Additional glycerol oxidases suitable for use in the present disclosure may be sourced from Neurospora and Penicillium.

As will be understood by one of ordinary skill in the art with the benefit of the present disclosure, reactions of the type disclosed herein may result in the production of byproducts (e.g., hydrogen peroxide) that can detrimentally impact other components of the reaction mixture. For example, hydrogen peroxide is a by-product of the GAO catalyzed oxidations. Hydrogen peroxide can degrade the biocatalyst resulting in a loss of catalytic activity which can mitigated by the introduction of a catalase (E.C. 1.11.1.61). In some aspects, the effect of hydrogen peroxide on the catalyst in the GLYCAT is mitigated. For example, mitigation of the effects of hydrogen peroxide on the GLYCAT may be through using any suitable mechanism such as the introduction of a catalase to the reaction media, through the use of a hydrogen peroxide-resistant biocatalyst, or through recovery of the hydrogen peroxide.

In an aspect, a GLYCAT is reacted in the presence of one or more cofactors. Herein a cofactor refers to non-protein chemical compound that modulates the biological activity of the biocatalyst. Nonlimiting examples of enzyme cofactors suitable for use in the present disclosure include thiamine pyrophosphate, NAD+, NADP+, pyridoxal phosphate, methyl cobalamin, cobalamine, biotin, Coenzyme A, tetrahydrofolic acid, menaquinone, ascorbic acid, flavin mononucleotide, flavin adenine dinucleotide, and Coenzyme F420. Such cofactors may be included in the GLYCAT and/or be added at various points during the reaction. In some aspects, cofactors included with the biocatalyst preparation may be readily regenerated with oxygen and/or may remain stable throughout the lifetime of the enzyme(s).

In one or more aspects, the GLYCAT is a wild type enzyme, a functional fragment thereof or a functional variant thereof. “Fragment” as used herein is meant to include any amino acid sequence shorter than the full-length biocatalyst (e.g., AOX), but where the fragment maintains a catalytic activity sufficient to meet some user or process goal. Fragments may include a single contiguous sequence identical to a portion of the biocatalyst sequence. Alternatively, the fragment may have or include multiple (e.g., several) different shorter segments where each segment is identical in amino acid sequence to a different portion of the amino acid sequence of the biocatalyst but linked via amino acids differing in sequence from the biocatalyst. Herein, a “functional variant” of the biocatalyst refers to a polypeptide which has at one or more positions of an amino acid insertion, deletion, or substitution, either conservative or non-conservative, and wherein each of these types of changes may occur alone, or in combination with one or more of the others, one or more times in a given sequence but retains catalytic activity.

In the alternative or in combination with the aforementioned mutations, the GLYCAT may be mutated to improve the catalytic activity. Mutations may be carried out in order to enhance the protein or a homolog activity, increase the protein stability in the presence of glycolaldehyde and/or hydrogen peroxide, increase protein yield, or combinations thereof.

Herein, reference has been made to “sources” of biocatalysts. It is to be understood this refers to the biomolecule as expressed by the named organism. It is contemplated the biocatalyst may be obtained from the organism or a version of said biocatalyst (wildtype or recombinant) provided as a suitable construct to an appropriate expression system.

In an aspect, any biocatalyst of the type disclosed herein may be cloned into an appropriate expression vector and used to transform cells of an expression system such as E. coli, Saccharomyces sp., Pichia sp., Aspergillus sp., Trichoderma sp., or Myceliophthora sp. As used herein, “vector” refers to a replicon, such as plasmid, phage, viral construct or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more AOX gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the gene of a biocatalyst disclosed herein is provided as a recombinant sequence in a vector where the sequence is operatively linked to one or more control or regulatory sequences. “Operatively linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences that are necessary to affect the expression of coding sequences to which they are operatively linked. Expression control sequences are sequences that control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences may differ depending upon the host organism; for example, in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

Methods and compositions of the present disclosure, in one or more aspects, generate glyoxal at high purity using an in-vitro system. For example, the glyoxal product may have a purity of equal to or greater than about 70%, alternatively equal to or greater than about 80% or alternatively equal to or greater than about 90% or alternatively from about 70% to about 95%. In one or more aspects, the high purity glyoxal product is obtained using a procedure that does not include ion exchange.

In one or more aspects, the GLYCAT results in yields of glyoxal ranging from about 50% to about 100%, additionally or alternatively, from about 60% to about 95% or, additionally or alternatively, from about 70% to about 90%.

Disclosed herein are methods using purified or partially purified enzymes as catalysts. The methods of the present disclosure facilitate the separation and purification of the glyoxal and lead to lower production costs. In one or more aspects, the methods disclosed herein utilizing enzymatic catalysts reduces the use of hazardous chemicals typically employed in the process for production of glyoxal such as formaldehyde, nitric acid, or formic acid. Advantageously, the present disclosure may use as a starting material an ethylene glycol feedstock derived from petrochemical sources, or an ethylene glycol feedstock derived from renewable resources providing a pathway to a greener process than conventional methods.

### Additional Disclosure

The following are nonlimiting aspects of the presently disclosed subject matter:

A first aspect which is a chemoenzymatic process comprising contacting ethylene glycol with a biocatalyst in the presence of oxygen under conditions suitable for the formation of glycolaldehyde, glyoxal or combinations thereof.

A second aspect which is the process of the first aspect wherein the biocatalyst is selected from the group consisting of a copper radical oxidase, an alcohol oxidase, a glycerol oxidase, and combinations thereof.

A third aspect which is the process of any of the first through second aspects wherein the catalyst comprises a copper radical oxidase.

A fourth aspect which is the process of the third aspect wherein the copper radical oxidase comprises galactose oxidase.

A fifth aspect which is the process of any of the third through fourth aspects wherein the contacting is carried out in the presence of a small molecule activator.

A sixth aspect which is the process of any of the first through fifth aspects wherein the contacting is carried out in the presence horseradish peroxidase.

A seventh aspect which is the process of any of the fifth through sixth aspects wherein the small molecule activator is selected from the group consisting of L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3 (4H)-one, 2-oxindole, beta-lapachone, menaquinone, thiamine, 4-methyl-5-thiazoleethanol, disodium 6-hydroxy-5-((2-methoxy-5-methyl-4-sulfophenyl) azo)-2-naphthalenesulfonate (Allura Red AC), menadione, p-cresol, ethyl-[4-[[4-[ethyl-[(3-sulfophenyl)methyl]amino]phenyl]-(4-hydroxy-2-sulfophenyl)methylidene]-1-cyclohexa-2,5-dienylidene]-[(3-sulfophenyl)methyl]azanium (Fast green FCF), disodium α-(4-(N-ethyl-3-sulfonatobenzylamino)phenyl)-α-(4-N-ethyl-3-sulfonatobenzylamino)cyclohexa-2,5-dienylidene) toluene-2-sulfonate (Brilliant Blue FCF), methylisothiazolinone, caffeine, veratryl alcohol, fluorescein, and combinations thereof.

An eighth aspect which is the process of any of the first through seventh aspects wherein the biocatalyst has SEQ ID. NO 1.

A ninth aspect which is the process of any of the first through eighth aspects wherein the contacting is carried out in the presence of a catalase.

A tenth aspect which is the process of any of the first through ninth aspects wherein the glycolaldehyde, glyoxal or both have a purity of equal to or greater than about 70%.

An eleventh aspect which is the process of any of the first through tenth aspects wherein glyoxal has a yield of from about 50% to about 100%.

A twelfth aspect which is a method of producing glyoxal comprising contacting ethylene glycol with an oxidase selected from the group consisting of a copper radical oxidase, an alcohol oxidase, a glycerol oxidase, and combinations thereof in the presence of oxygen and a catalase under conditions suitable for the formation of a product mixture comprising glycolaldehyde, glyoxal or combinations thereof.

A thirteenth aspect which is the method of the twelfth aspect wherein the oxidase comprises a copper radical oxidase.

A fourteenth aspect which is the method of any of the twelfth through thirteenth aspects wherein the copper radical oxidase is a galactose oxidase having SEQ ID NO 1.

A fifteenth aspect which is the method of any of the twelfth through fourteenth aspects wherein the contacting is carried out in the presence of a small molecule activator.

A sixteenth aspect which is the method of any of the twelfth through fifteenth aspects wherein the small molecule activator is selected from the group consisting of L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3 (4H)-one, 2-oxindole, beta-lapachone, menaquinone, thiamine, 4-methyl-5-thiazoleethanol, disodium 6-hydroxy-5-((2-methoxy-5-methyl-4-sulfophenyl) azo)-2-naphthalenesulfonate (Allura Red AC), menadione, p-cresol, ethyl-[4-[[4-[ethyl-[(3-sulfophenyl)methyl]amino]phenyl]-(4-hydroxy-2-sulfophenyl)methylidene]-1-cyclohexa-2,5-dienylidene]-[(3-sulfophenyl)methyl]azanium (Fast green FCF), disodium α-(4-(N-ethyl-3-sulfonatobenzylamino)phenyl)-α-(4-N-ethyl-3-sulfonatobenzylamino)cyclohexa-2,5-dienylidene) toluene-2-sulfonate (Brilliant Blue FCF), methylisothiazolinone, caffeine, veratryl alcohol, fluorescein, and combinations thereof.

A seventeenth aspect which is the method of any of the twelfth through sixteenth aspects wherein the oxidase is a glycerol oxidase.

An eighteenth aspect which is the method of any of the twelfth through seventeenth aspects wherein the oxidase is an alcohol oxidase.

A nineteenth aspect which is the method of any of the twelfth through eighteenth aspects wherein the contacting occurs in a bubble reactor.

A twentieth aspect which is the method of any of the twelfth through nineteenth aspects wherein the glycolaldehyde, glyoxal or both have a yield of from about 50% to about 100%.

### EXAMPLES

The subject matter having been generally described, the following examples are given as particular aspects of the disclosure and are included to demonstrate the practice and advantages thereof, as well as aspects and features of the presently disclosed subject matter. It should be appreciated by those of skill in the art that the techniques disclosed in the examples which follow represent techniques discovered by the inventors to function well in the practice of the present subject matter, and thus can be considered to constitute preferred modes for its practice. However, those of skill in the art should, in light of the present disclosure, appreciate that many changes can be made in the specific aspects which are disclosed and still obtain a like or similar result without departing from the scope of the instant disclosure. It is understood that the examples are given by way of illustration and are not intended to limit the specification of the claims to follow in any manner.

### Example 1

### Development of Purpald Assay for the Quantification of Glyoxal and Glycolaldehyde in GAO Catalyzed Ethylene Glycol (EG) Oxidation Reactions

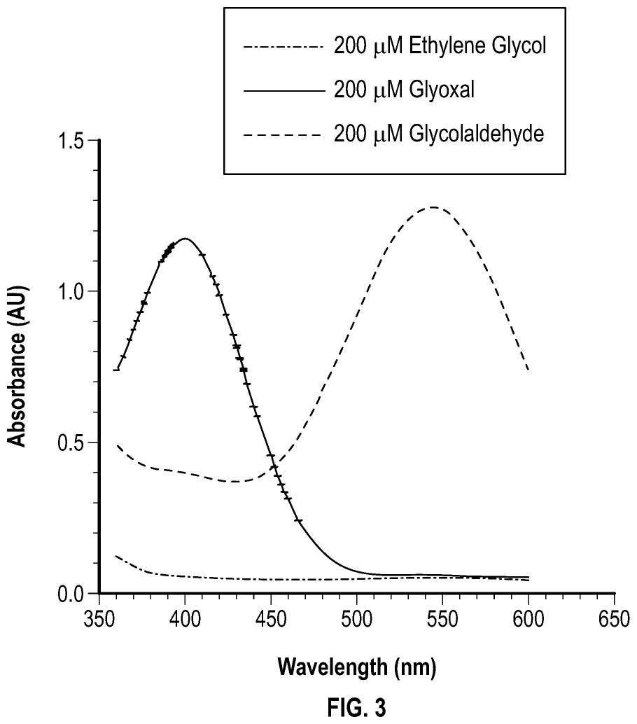

GAO engineering was carried out to obtain higher ethylene glycol conversion, e.g., by high throughput screening of GAO mutation libraries. A rapid method to quantify the product was developed. A chromogenic method, the Purpald assay, was developed to rapidly (greater than 600 samples/hour) and accurately quantify the amount of glyoxal and glycolaldehyde. Glycolaldehyde and glyoxal was reacted with 100 mM Purpald reagent (4-amino-3-hydrazino-5-mercapto-1,2,4-triazole) in 1 M NaOH solution via their aldehyde group(s) and formed purple and yellow color products after oxidation by dioxygen (O₂) in the ambient air. Since ethylene glycol does not contain any aldehyde groups, it does not react with the Purpald reagent. Wavelength scan analysis showed that the products of glycolaldehyde and glyoxal have maximum absorption at 550 nm and 400 nm, respectively. The results are depicted in FIG. 3. The absorbance values were found to correlate linearly with the concentrations of starting materials ranging between 20 UM and 200 UM). Further, the product of glyoxal did not have any absorbance at 550 nm (an “A550 value) though the product of glycolaldehyde has absorbance at 400 nm (an “A400 value”). Therefore, the A550 value can be used to quantify the glycolaldehyde in a mixture of ethylene glycol, glycolaldehyde and glyoxal such as reaction samples from the GAO and ethylene glycol. Nevertheless, the combination of A550 and A400 values can be used to quantify glyoxal in the same mixture. This is validated by the quantification of glycolaldehyde and glyoxal in their mixtures with known concentrations and comparison of quantification data from Purpald assay and those from HPLC-RI analysis.

### Example 2

### Demonstration of Glyoxal Production Using Engineered Galactose Oxidase at 4 Liter Scale

An engineered galactose oxidase, GAO-Mut327 (100 mg/L), was reacted with 2.22 percentage weight by volume w/v % ethylene glycol in 50 mM sodium phosphate pH 7.5, 100 mg/L catalase, 3 mg/L horseradish peroxidase, 92 UM copper nitrate, 48 UM manganese nitrate, 5 ppm of a small molecule activator, and 0.05% antifoam. The reaction was conducted over a period of seven hours in the presence of pure oxygen (250 ml/min flow rate) at 11 °C, 8 bar (116 psi). Following seven hours of stirred incubation, the reaction produced 1.63 weight percent (wt. %) glyoxal and glycolaldehyde (0.022 wt. %). Concentrations of glyoxal, EG, and glycolaldehyde were monitored using the Purpald assay as described in Example 1 and final titers cross validated with a quantitative HPLC-RI method preceding and following concentration.

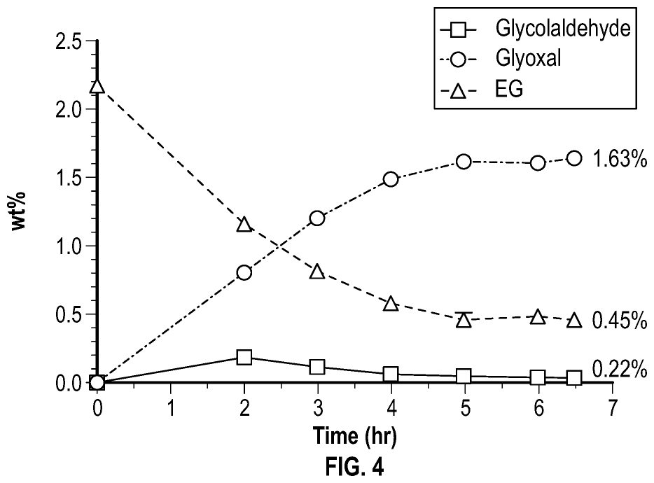

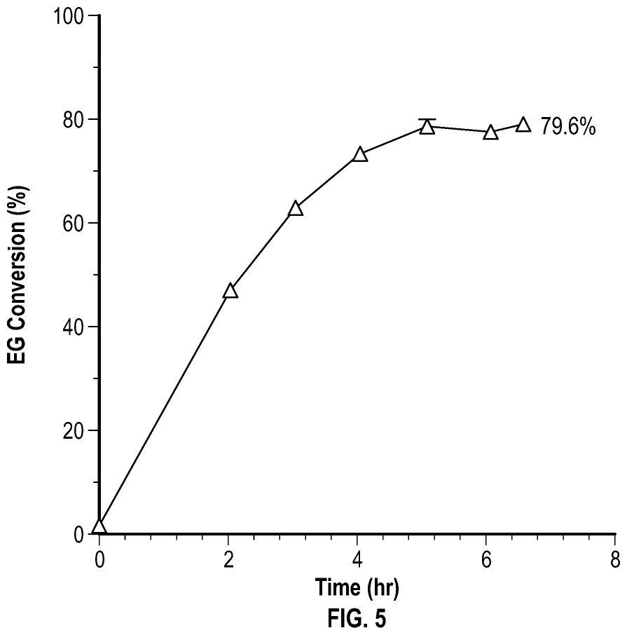

The concentrations of glyoxal, glycolaldehyde and ethylene glycol (EG) were plotted as a function of time and the results are presented in FIG. 4. A final conversion of 79.6% was achieved resulting in a residual 0.45 wt. % of EG, this is depicted in FIG. 5. The product was concentrated via rotoevaporation to a final glyoxal content of 43.5 wt. % with a pH of 2.91.

The subject matter having been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A chemoenzymatic process comprising:
contacting ethylene glycol with a biocatalyst in the presence of oxygen under conditions suitable for the formation of glycolaldehyde, glyoxal, or combinations thereof.

2. The process of claim 1, wherein the biocatalyst is selected from the group consisting of a copper radical oxidase, an alcohol oxidase, a glycerol oxidase, and combinations thereof.

3. The process of claim 1, wherein the catalyst comprises a copper radical oxidase.

4. The process of claim 3, wherein the copper radical oxidase comprises galactose oxidase.

5. The process of claim 3, wherein the contacting is carried out in the presence of a small molecule activator.

6. The process of claim 5, wherein the contacting occurs in the presence of horseradish peroxidase.

7. The process of claim 5, wherein the small molecule activator is selected from the group consisting of L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3 (4H)-one, 2-oxindole, beta-lapachone, menaquinone, thiamine, 4-methyl-5-thiazoleethanol, disodium 6-hydroxy-5-((2-methoxy-5-methyl-4-sulfophenyl) azo)-2-naphthalenesulfonate (Allura Red AC), menadione, p-cresol, ethyl-[4-[[4-[ethyl-[(3-sulfophenyl)methyllamino]phenyl]-(4-hydroxy-2-sulfophenyl)methylidene]-1-cyclohexa-2,5-dienylidene]-[(3-sulfophenyl)methyl]azanium (Fast green FCF), disodium α-(4-(N-ethyl-3-sulfonatobenzylamino)phenyl)-α-(4-N-ethyl-3-sulfonatobenzylamino)cyclohexa-2,5-dienylidene) toluene-2-sulfonate (Brilliant Blue FCF), methylisothiazolinone, caffeine, veratryl alcohol, fluorescein, and combinations thereof.

8. The process of claim 1, wherein the biocatalyst has SEQ ID. NO 1.

9. The process of claim 1, wherein the contacting is carried out in the presence of a catalase.

10. The process of claim 1, wherein the glycolaldehyde, glyoxal or both have a purity of equal to or greater than about 70%.

11. The process of claim 1, wherein the glycolaldehyde, glyoxal or both have a yield of from about 50% to about 100%.

12. A method of producing glyoxal comprising:
contacting ethylene glycol with an oxidase selected from the group consisting of a copper radical oxidase, an alcohol oxidase, a glycerol oxidase, and combinations thereof in the presence of oxygen and a catalase under conditions suitable for the formation of a product mixture comprising glycolaldehyde, glyoxal or combinations thereof.

13. The method of claim 12, wherein the oxidase comprises a copper radical oxidase.

14. The method of claim 12, wherein the copper radical oxidase is a galactose oxidase having SEQ ID NO 1.

15. The method of claim 12, wherein the contacting is carried out in the presence of a small molecule activator.

16. The method of claim 12, wherein the small molecule activator is selected from the group consisting of L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3 (4H)-one, 2-oxindole, beta-lapachone, menaquinone, thiamine, 4-methyl-5-thiazoleethanol, disodium 6-hydroxy-5-((2-methoxy-5-methyl-4-sulfophenyl) azo)-2-naphthalenesulfonate (Allura Red AC), menadione, p-cresol, ethyl-[4-[[4-[ethyl-[(3-sulfophenyl)methyllamino]phenyl]-(4-hydroxy-2-sulfophenyl)methylidene]-1-cyclohexa-2,5-dienylidene]-[(3-sulfophenyl)methyl]azanium (Fast green FCF), disodium α-(4-(N-ethyl-3-sulfonatobenzylamino)phenyl)-α-(4-N-ethyl-3-sulfonatobenzylamino)cyclohexa-2,5-dienylidene) toluene-2-sulfonate (Brilliant Blue FCF), methylisothiazolinone, caffeine, veratryl alcohol, fluorescein, and combinations thereof.

17. The method of claim 12, wherein the oxidase is a glycerol oxidase.

18. The method of claim 12, wherein the oxidase is an alcohol oxidase.

19. The method of claim 12, wherein the contacting occurs in a bubble reactor.

20. The method of claim 12, wherein the glycolaldehyde has a yield of from about 50% to about 100%.


SEQUENCE LISTING (1 sequence)

SEQ ID NO: 1 - protein, 657 aa, synthetic construct
MGHHHHHHSSGHIEGRHMASAPIGSAIPRNNWAVTCDSAQSGNECNKAIDGNKDTFWHTFYGANGDPKPPHTYTIDMKTTQNVSGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPVAAAIEPTSGRVLMWSSYGNDRFEGSPGGITLTSSWDPSTGIVSDRTVTNTKHDMFCPGISMDGNGQIVVTGGNDAKKTSLYDSSSDSWIPGPDMQVARGYQSSATMSDGRVFTIGGSFSGGVFEKNGEVYSPSAKTWTFLPNAKVEPMLTADKQGLFRSDNHAWLFGWKKGSVFQAGPSTAMNWYYTSGSGDVKSAGKRQSNRGVADDQYSGNAVMYDAVKGKILTFGGAPHFEDSDATTNAHIITLGEPGTSPATVFASNGLYFARTFHTSVVLPDGSTFITGGQRRGEPFKDSYPVLTPEIYVPEQDTFYKLNPNSIVRVYHSISLLLPDGRVFNGGGGLCGDCTTNHFDAQIFTPNYLYDANGNFATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ